SEQUENCE LISTING (3 sequences)

SEQ ID NO: 1 - DNA, 8 bp, Hominidae
tgacgtca

SEQ ID NO: 2 - DNA, 26 bp, Hominidae
agcttcagaggggactttccgagagg

SEQ ID NO: 3 - DNA, 30 bp, Hominidae
agcttcagaggggactttccgagaggtcga

(19) United States
(12) Patent Application Publication
(10) Pub. No.: US 2004/0048791 A1
(43) Pub. Date: Mar. 11, 2004

(54) Method for treating neoplastic cells using cycloprodigiosin or salt thereof

(76) Inventors: Keiko Kamata, Hyogo (JP); Hajime Hirata, Hyogo (JP); Yoshinari Ikegami, Hyogo (JP); Kouji Nakagawa, Hyogo (JP)

Correspondence Address: KNOBBE MARTENS OLSON & BEAR LLP, 2040 MAIN STREET, FOURTEENTH FLOOR, IRVINE, CA, 92614, US

(21) Appl. No.: 10/237,588

(22) Filed: Sep. 5, 2002

(51) Int. Cl. A61K 38/17

(52) U.S. Cl. 514/12

(57) ABSTRACT

Neoplastic cells are treated by examining whether a potential subject has neoplastic cells in which Rb is present and NF-κB and/or AP-1 are constitutively being activated and then administering cycloprodigiosin or a pharmaceutically acceptable salt thereof in an amount effective to suppress Rb phosphorylation and to suppress transcriptional activity of NF-κB and/or AP-1 which are constitutively being activated, to the potential subject. Alternatively, an autoimmune disease is treated by examining whether a potential subject has said autoimmune disease caused by gene expression which depends on binding to the CRE sequence and then administering cycloprodigiosin or a pharmaceutically acceptable salt thereof in an effective amount to said potential subject.

### BACKGROUND OF THE INVENTION

1. Field of the Invention

The present invention relates to a method of treating neoplastic cells using cycloprodigiosin or a pharmaceutically acceptable salt thereof, and in particular to a method of treating tumor cells in which cell cycle and apoptosis control are obstructed. Further, the present invention related to a method of treating an autoimmune disease using cycloprodigiosin or a pharmaceutically acceptable salt thereof, and in particular to a method of treating an autoimmune disease which is caused by gene expression depending on binding to the CRE sequence.

2. Description of the Related Art

Cycloprodigiosin hydrochloride (cPrG.HCl) produced by marine bacteria is a H⁺/Cl⁻ symporter or a physiologically active substance which induces acidification of intracellular pH. To date the present inventors analyzed the effect of cPrG.HCl on activation of transcription factors NF-κB and AP-1, which play an important role in cell growth or the like, and found that cPrG.HCl showed no suppressive effect on IKK activation, IκB degradation, or nuclear translocation of NF-κB as well as on JNK activation but significantly suppressed gene expression induction by NF-κB and AP-1 (Keiko Kamata, et al., “Suppression of NF-κB and AP-1 Activation by Cycloprodigiosin,” September 2000, Abstract, The Japanese Association of Biochemical Society). Further, the present inventors applied a patent for an anticancer agent having cPrG as an effective component (Japanese Patent Application Laid-open No. H11-209283, published on Aug. 3, 1999).

On the other hand, it has been reported that NF-κB activation by TNFα induces gene expression of the viability enhancing gene of cells, such as the IAP gene (Wang, C. Y., Mayo, M. W., Komeluk, R. G., Goeddel, D. V., Baldwin, A. S., Jr. (1998) Science 281:1680-1683). It has also been revealed that when NF-κB activity is contrarily suppressed, cell death by TNFα is markedly enhanced (Beg, A. A., Baltimore, D. (1996) Science 274:782-784; Chu, Z. L., McKinsey, T. A., Lin, L., Gentry, J. J., Malim, M. H., Ballard, D. W. (1997) Proc Natl Acad Sci USA 94:10057-10062; Kitahara, J., Sakamoto, H., Tsujimoto, M., Nakagawa, Y. (2000) Biol. Pharm. Bull. 23:397-401; Van Antwerp, D. J., Martin, S. J., Kafri, T., Green, D. R., Verma, I. M. (1996) Science 274:787-789).

Further, it has been revealed that inflammatory cytokines IL-6 and IL-8 involve in noninfectious inflammatory diseases represented by chronic articular rheumatism (Feldmann, M., Maini, R. N. (2001) Annu. Rev. Immunol. 19:163-196); however, their relation to cPrG has not been revealed.

### SUMMARY OF THE INVENTION

As mentioned above, cPrG has already been reported by the present inventors as a possible anticancer agent. However, it is totally uncertain whether cPrG significantly enhances apoptosis and is clinically applicable for treating tumor cells even if it is proved to suppress transcriptional activation of NF-κB and/or AP-1, since substances such as N-acetylcysteine which inhibit NF-κB and inhibits apoptosis are known (Oka, S., Kamata, H., Kamata, K., Yagisawa, H. and Hirata, H. (2000) FEBS Lett. 472:196-202). Further, not only cell cycle but also apoptosis control is generally obstructed in tumor cells and thus it is not certain whether cPrG can be effective as an anticancer agent only with its suppressive action on transcriptional activation of NF-κB and/or AP-1.

The present inventors intensively studied to complete the present invention. Namely, according to one embodiment of the present invention, there is provided a method of treating neoplastic cells comprising the steps of examining whether a potential subject has neoplastic cells in which Rb is present and NF-κB and/or AP-1 are constitutively being activated and administering cycloprodigiosin (cPrG) or a pharmaceutically acceptable salt thereof in an amount effective to suppress Rb phosphorylation and to suppress transcriptional activity of NF-κB and/or AP-1, which are constitutively being activated, to the potential subject to treat the neoplastic cells. According to this embodiment, an unprecedented therapeutic effect can be attained since cPrG (or a salt thereof) suppresses Rb phosphorylation to deal with abnormal cell cycle and suppresses transcriptional activity of NF-κB and/or AP-1 to deal with abnormal apoptosis control, which results in amelioration of abnormal growth of neoplastic cells in terms of both aspects mentioned above.

According to one embodiment, said neoplastic cells are tumor cells selected from the group consisting of leukemic cells, testic cancer, melanoma, breast cancer, and hepatoma.

Said pharmaceutically acceptable salt is hydrochloride according to one embodiment.

Further, in another embodiment, said neoplastic cells are effectively treated in the presence of tumor necrosis factor. For example, the tumor necrosis factor is TNFα. The tumor necrosis factor effectively acts on cells, in which transcriptional activity of NF-κB and/or AP-1 is suppressed, and thus apoptosis can be enhanced. Tumor necrosis factor such as TNFα can also be administered together with cycloprodigiosin.

Further, according to another embodiment of the present invention, there is provided a method of treating neoplastic cells comprising the

steps of examining whether a potential subject has neoplastic cells in which Rb is present and NF-κB and/or AP-1 are not being constitutively activated and administering cycloprodigiosin or a pharmaceutically acceptable salt thereof in an amount effective to suppress Rb phosphorylation, to the potential subject to treat the neoplastic cells. According to this embodiment, malignant alteration of neoplastic cells can be prevented by suppressing phosphorylation of the Rb gene by cPrG (or a salt thereof) to ameliorate abnormal cell cycle and allow the cancer suppressing gene function even if NF-κB and/or AP-1 are not constitutively being activated. Cells in which NF-κB and/or AP-1 are not being constitutively activated include both cells in which they are not being naturally activated and cells in which they can be activated but are still in the preceding stage. Examples of such cells are selected from the group consisting of cancerous tissues of colon cancer, rectal cancer, uterine cancer, and skin cancer, leukemia and the like, except those in the abovementioned constitutively activated stage.

Further, the present inventors found that autoimmune diseases including rheumatism can be treated by inhibiting gene expression depending on a transcription factor and binding to a CRE sequence and NF-κB- or AP-1-dependent gene expression of cytokines or chemoatractants, by cPrG-HCl. Namely, according to another embodiment of the present invention, there is provided a method of treating an autoimmune disease which is caused by gene expression of a cytokine or chemoatractant (or cell adhesive factor) depending on binding to the CRE sequence and NF-κB- or AP-1-dependent gene expression. This is a method of treating an autoimmune disease comprising the steps of examining whether a potential subject has an autoimmune disease caused by gene expression depending on binding to the CRE sequence and administering an effective amount of cycloprodigiosin or a pharmaceutically acceptable salt thereof to the potential subject to treat the autoimmune disease.

The pharmaceutically acceptable salt is hydrochloride according to one embodiment.

Further, an example of the gene binding to the CRE sequence is a gene of an active factor selected from the group consisting of cytokines, chemoatractants and cell adhesive factors. Further, an example of the autoimmune disease is rheumatism.

For purposes of summarizing the invention and the advantages achieved over the prior art, certain objects and advantages of the invention have been described above. Of course, it is to be understood that not necessarily all such objects or advantages may be achieved in accordance with any particular embodiment of the invention. Thus, for example, those skilled in the art will recognize that the invention may be embodied or carried out in a manner that achieves or optimizes one advantage or group of advantages as taught herein without necessarily achieving other objects or advantages as may be taught or suggested herein.

Further aspects, features and advantages of this invention will become apparent from the detailed description of the preferred embodiments which follow.

### BRIEF DESCRIPTION OF THE DRAWINGS

These and other features of this invention will now be described with reference to the drawings of preferred embodiments which are intended to illustrate and not to limit the invention.

FIG. 1A is a graph showing apoptosis by cPrG-HCl in HeLa cells stimulated with TNFα.

FIG. 1B is a graph showing change in caspase-3 activity which plays a central role upon apoptosis induction.

FIG. 2A is a graph showing relationship between TNFα-induced NF-κB activation and the amount of cPrG-HCl administered.

FIG. 2B is a graph showing expressibility of the luciferase gene depending on the CMV promoter used as a control, by cPrG-HCl

FIG. 2C is a graph showing change in TNFα-induced NF-κB activation in the presence of imidazole, and change in TNFα-induced NF-κB activation when acidification of intracellular pH is induced by a V-ATPase inhibitor, bafilomycin.

FIG. 2D demonstrates that TNFα-induced IKK activation is not suppressed.

FIG. 2E shows Western blots showing the effect of cPrG-HCl on phosphorylation and degradation of IκB.

FIG. 2F shows Western blots of p65 and p50, which are NF-κB components, showing nuclear translocation of NF-κB.

FIG. 2G is the result of an EMSA (electrophoretic mobility shift assay) showing the state of activation of NF-κB in the nucleus.

FIG. 3A is a graph showing suppression of NF-κB activity by cPrG-HCl and PMA.

FIG. 3B is the result of an EMSA (electrophoretic mobility shift assay) showing the state of nuclear translocation by cPrG-HCl and PMA.

FIG. 4A, FIG. 4B and FIG. 4C are graphs showing the results of measuring the effect of cPrG-HCl on NF-κB activation by luciferase assay, in which an activated Rac1, Cdc42 or MEKKI expression plasmid was introduced into cells together with the reporter gene of NF-κB.

FIG. 5 is a graph showing relationship between cPrG-HCl and AP-1 transcriptional activity.

FIG. 6A and FIG. 6E are graphs, each showing suppression of transcriptional activity of NF-κB or AP-1 by cPrG-HCl in the 293 cells (human embryo-derived kidney cell).

FIG. 6B and FIG. 6F are graphs, each showing suppression of transcriptional activity of NF-κB or AP-1 by cPrG-HCl in the U373 cells (human brain tumor cell).

FIG. 6C and FIG. 6D are graphs, each showing suppression of transcriptional activity of NF-κB or AP-1 by cPrG-HCl in the COS7 cells (African green monkey kidney-derived cell) or the MKN45 cells (human gastric cancer cell).

FIG. 7 is a graph showing suppression of TNFα-induced IL-8 promoter-dependent transcriptional activity by cPrG-HCl.

FIG. 8 is a graph showing suppression of PMA-induced CRE-dependent transcriptional activity by cPrG-HCl.

FIG. 9A and FIG. 9B are Western blots, each showing no suppression on JNK kinase or p38 kinase by cPrG-HCl.

FIG. 10 is a Western blot showing Rb phosphorylation suppression by cPrG-HCl.

FIG. 11 is the result of experiment for cell labeling using [³²P] orthophosphoric acid, showing suppression of TNFα-induced phosphorylation by cPrG-HCl, for p65 phosphorylation (Image Analyzer analysis)

FIG. 12 is a graph showing that cPrG-HCl does not directly suppress casein kinase II (CKII) activity.

FIG. 13 shows that cPrG-HCl does not directly suppress PKA activity.

FIG. 14 shows that cPrG-HCl does not directly suppress IκB kinase (IKK) activity.

### DETAILED DESCRIPTION OF THE PREFERRED EMBODIMENT

Cycloprodigiosin used in the present invention is a compound represented by the following formula (I).

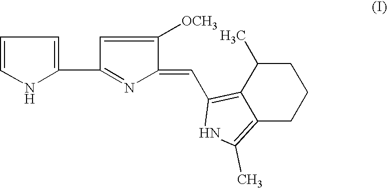

Cycloprodigiosin is a red pigment produced from a marine bacterium Alteromonas rubra (Laatsh, H., Thomson, R. H. (1983) Tetrahedron Lett. 24:2701-04) or Benekea gazogenes (Gerber, N. N. (1983) Tetrahedron Lett. 24: 2797-98), and is known to be synthesized using 2-methylcyclohexanone as a starting material (Wasserman, H. H., Fukuyama, J. M. (1984) Tetrahedron Lett. 25:1387-88). Since cycloprodigiosin extract obtained from these marine bacteria contains various kinds of prodigiosins as impurities, a simple cycloprodigiosin can be obtained by isolation from the extract. Further, cycloprodigiosin can be produced by chemical synthesis. In one embodiment, cycloprodigiosin hydrochloride containing no other prodigiosin impurities can be obtained from Pseudoalteromonas denitrificans AK-1 strain (FERM P-15771) (Japanese Patent Application Laid-open No. H10-080293, published on Mar. 31, 1998 and Japanese Patent Application Laid-open No. H11-209283, published on Aug. 3, 1999; the contents of disclosure of these publications are herein cited as references). If the abovementioned bacterial cells are cultured in this manner, cycloprodigiosin hydrochloride can be produced and accumulated in a large amount in the resulting cells and culture medium so that it can be abundantly obtained in a highly pure form. Free cycloprodigiosin can be obtained by readily removing hydrogen chloride using column chromatography. The abovementioned salt can be obtained by treating this free cycloprodigiosin (cycloprodigiosin hydrochloride from which hydrogen chloride is removed, i.e., a compound represented by formula (I)) with an acid such as phosphoric acid and sulfuric acid.

Further, examples of the pharmaceutically acceptable salt of cycloprodigiosin include hydrochloride, phosphate, sulfate, perchlorate, carboxylates (e.g., acetate, citrate, and tartarate), iodate, bromate, picrate, sulfonate, and tetrafluoroborate. The abovementioned compound such as cycloprodigiosin hydrochloride can be obtained by converting cycloprodigiosin into a salt form. The ordinary method for salt formation can be used to prepare these salts. Hydrochloride and other salts of cycloprodigiosin used in the present invention are water soluble and can be used by dissolving in distilled water or the like. Accordingly, they can be used as injections, health drinks, and the like. The hydrochloride can be represented by the following formula:

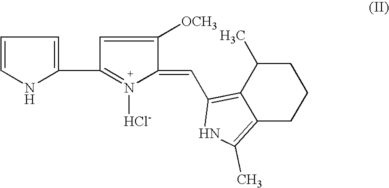

Treatment of Neoplastic Cells with cPrG (or its Salt)

According to an embodiment of the present invention, cPrG (or its salt) can be used for neoplastic cells in which Rb is present and NF-κB and/or AP-1 are constitutively being activated. The Rb gene (retinoblastoma gene) is one of cancer suppressor genes and cPrG can effectively suppresses phosphorylation of Rb to enhance cancer suppression. cPrG suppresses phosphorylation of not only Rb but also transcription factors NF-κB and AP-1; for example, it does not suppress nuclear translocation of NF-κB. However, cPrG translocates to the nucleus and then suppresses transcriptional activity of NF-κB binding to the gene; as a result, cPrG can block the action of NF-κB and thus enhances apoptosis. On the other hand, by suppressing phosphorylation of Rb by cPrG, cancerous changes in cells can be effectively prevented. Accordingly, cPrG is particularly effective on neoplastic cells in which Rb is present and NF-κB and/or AP-1 are constitutively being activated.

Rb is found to be inactivated in about 20% of whole cancerous cells in pulmonary small cell cancer, breast cancer and the like other than retinoblastoma; Rb is also present in various cells such as human genital epidermoid carcinoma A431 cells (Ahmad, N., Gupta, S., Mukhtar, H. (1999) Oncogene 18:1891-6; Bio Science Term Library, edited by Yoichi Taya and Masashi Yamamoto (1997) 116-7). Further, NF-κB and/or AP-1 are constitutively being activated in leukemic cells, testis cancer, melanoma, breast cancer, hepatoma (Ni, H., Ergin, M., Huang, Q., Qin, J. Z., Amin, H. M., Martinez, R. L., Saeed, S., Barton, K., Alkan, S. (2001) Br. J. Haematol. 115: 279-86; Rath, P. C., Aggarwal, B. B. (2001) J. Interferon Cytokine Res. 21: 523-8; Chen, C. D., Sawyers, C. L. (2002) Mol. Cell Biol. 22: 2862-70; Meyskens, F. L., Jr., Buckmeier, J. A., McNulty, S. E., Tohidian, N. B. (1999) Clin. Cancer Res.5: 1197-202; Nakshatri, H., Bhat-Nakshatri, P., Martin, D. A., Goulet, R. J., Jr., Sledge, G. W., Jr. (1997) Mol. Cell Biol. 17:3629-39; Mukhopadhyay, A., Bueso-Ramos, C., Chatterjee, D., Pantazis, P., Aggarwal, B. B. (2001) Oncogene 20:7597-609; Tai, D. I., Tsai, S. L., Chang, Y. H., Huang, S. N., Chen, T. C., Chang, K. S., Liaw, Y. F. (2000) Cancer 89: 2274-81). Accordingly, by targeting neoplastic cells having these two factors, abnormality in both cell cycle and apoptosis control can be effectively attacked. A therapeutic effect much better than those attained by conventional antitumor agents can be realized by allowing cPrG to exhibit the abovementioned two actions. Without depending on the abovementioned literature, the presence of Rb in target cells can be found by a genetic diagnosis such as chromosomal analysis (test method), and whether NF-κB and/or AP-1 are being constitutively activated in the cells can be found by analyzing the activation state of NF-κB in the nucleus using EMSA (electrophoretic mobility shift assay) (test method).

Since cPrG acts to suppress transcriptional activity after NF-κB and/or AP-1 are bound to the DNA sequence, it is not very effective if administered in the state where NF-κB and/or AP-1 are not constitutively activated. Namely, its use for prevention is not so effective. In cancerous cells, for example, cPrG can suppress tumor necrosis factor TNFα-induced transcriptional activation by NF-κB, and enhance TNFα-induced apoptosis.

On the other hand, the suppressive effect on Rb phosphorylation exhibits an effect different from the effect on NF-κB and/or AP-1 activation. Accordingly, cPrG administration can be started when NF-κB and/or AP-1 are not being activated, namely in the early stage. Cells in which NF-κB and/or AP-1 are not being constitutively activated include both cells in which they are not being naturally activated and cells in which they can be activated but are still in the preceding stage.

When cPrG is administered in the state where NF-κB and /or AP-1 are being constitutively activated, in one embodiment, neoplastic cells can be effectively treated in the presence of tumor necrosis factor. For example, the tumor necrosis factor is TNFα (tumor necrosis factor α). The tumor necrosis factor effectively acts on cells, in which transcriptional activity of NF-κB and/or AP-1 is suppressed, and thus apoptosis can be enhanced. Tumor necrosis factor such as TNFα can also be administered together with cycloprodigiosin. In such a case, the tumor necrosis factor can be administered in a ratio of about 1 to 10 by weight to cPrG together or separately with cPrG (according to the method of administration of cPrG described below).

TNFα is a cytokine which is important in immune response and is produced by various cells such as macrophages and monocytes. It is now known that in many cells, no apoptosis is induced and the cells remain viable even when treated with TNFα. For a long time, the mechanism of this apoptosis suppression was not known; however, recently, it was revealed that apoptosis is suppressed by inducing the expression of the inhibitor of the apoptosis protein (IAP) gene or the like by NF-κB activated by TNFα (Wang, C. Y., Mayo, M. W., Komeluk, R. G., Goeddel, D. V., Baldwin, A. S., Jr. (1998) Science 281: 1680-3). Further, it has been reported that not only NF-κB but also AP-1 functions as a survival factor (Mukhopadhyay, A., Bueso-Ramos, C., Chatte jee, D., Pantazis, P., Aggarwal, B. B. (2001) Oncogene 20:7597-609).

cPrG can be used as it is; however, it is generally made into an appropriate pharmaceutical form together with an excipient and other auxiliary agents. Examples of oral agents include powders, granules, tablets, sugarcoated agents, pills, capsules, and health drinks. Parenteral agents include suppositories, pastes, and injections. The injections can be for intramuscular injection, intra-arterial injection, intravenous injection, or the like. In particular, subcutaneous injection is effective.

Examples of the excipient and other auxiliary agents include lactose, sucrose, various starches, glucose, cellulose or its derivatives, magnesium stearate, and talc. Further, a solvent such as physiological serine and distilled water can be used. Further, a surfactant for solubilization or an analgesic agent can be added. They can be formed into an oral or parenteral preparation by a generally known method for preparation. The amount of dose varies depending on symptoms, age, sex and the like; however, the daily dose of cPrG is preferably 0.1 to 100 mg, preferably 1 to 10 mg/kg body weight for oral administration and 0.1 to 50 mg, preferably 1 to 5 mg/kg body weight for injection for parenteral administration.

One example of the preparation is prepared by dissolving cPrG hydrochloride at a specified concentration, for example by dissolving 20 mg of cPrG-HCl and 0.4 g of Tween 80 in 200 ml of distilled water, dispensing the solution into 20-ml ampoules and sterilizing by heat to prepare an intravenous injection. The method is not limited to this example and any preparation method can be used.

As for toxicity of these compounds, when cPrG was intraperitoneally administered to female ICR mice (5 weeks old, 25 to 29 g of body weight), the LD50 was greater than 10 mg; when resistivity of cPrG was studied by the phospholipid plane membrane method (Sin-seikagaku Jikken Kouza “Biomembrane and Membrane Transformation, I,” 181-187, edited by Japan Biochemistry Society, published by Tokyo Kagaku Dojin, Jul. 10, 1992), the result showed that cPrG exhibited no ionophore activity as compared to carbonyl cyanide p-trifluoromethoxyphenylhydrazone (FCCP), one of ionophores, and thus was judged to be extremely low in toxicity.

Further, since cPrG suppresses phosphorylation of Rb and transcriptional activity of NF-κB and/or AP-1, its use with other anticancer agents (e.g., cisplatin), or with a therapeutic method other than chemotherapy (e.g., radiation, surgery) is extremely effective. Furthermore, cPrG is also highly effective on tumor cells which have acquired multi-drug resistance considering from its effectiveness.

Treatment of Autoimmune Disease by cPrG

Autoimmune diseases including rheumatism are presumably be caused by gene expression of a cytokine or chemoatractant (or cell adhesive factor), which depends on binding to the CRE sequence, and NF-κB- or AP-1-dependent gene expression. It is revealed that an inflammatory cytokine, IL-6 or IL-8, is associated with noninfectious inflammatory diseases represented by chronic articular rheumatism. In the present invention, cPrG inhibits gene expression depending on transcription factor and binding to the CRE sequence and NF-κB- or AP-1-dependent gene expression of a cytokine or chemoatractant, and can thus treat autoimmune diseases including rheumatism. Namely, the present invention provides a method of treating an autoimmune disease comprising the steps of diagnosing whether a potential subject has an autoimmune disease caused by gene expression depending on binding to the CRE sequence and administering an effective amount of cycloprodigiosin or a pharmaceutically acceptable salt thereof to the potential subject to treat the autoimmune disease.

The CRE is a cAMP response element (CRE) having a DNA sequence TGACGTCA (SEQ ID NO: 1) and a transcription factor called CREB/ATF family is bound to the CRE. This family members include ATF-2 and CREB. The ATF-2 forms a homodimer or a heterodimer and binds to the CRE. The ATF-2 forms a heterodimer with jun, which comprises a region phosphorylated by JNK or p38 and a transcriptional activation domain. When p38 is activated by an inflammatory cytokine (e.g., TNFα, IL-1β) or stress (e.g., UV, γ-ray radiation), the ATF-2 is phosphorylated and bound to the CRE. It has been revealed from studies using ATF-2 knockout mice that the production of inflammatory cytokines such as IL-6 is suppressed and that the CRE is present in the IL-8 gene (Reimold, A. M., Kim, J., Finberg, R., Glimcher, L. H. (2001) Int. Immunol. 13:241-8; Taylor, C. T., Fueki, N., Agah, A., Hershberg, R. M., Colgan, S. P. (1999) J. Biol. Chem. 274: 19447-54).

The abovementioned mode of usage and the like of cPrG for neoplastic cells are herein similarly applicable.

### EXPERIMENTAL EXAMPLE 1

Suppression of Transcriptional Activation of NF-κB Induced by Tumor Necrosis Factor TNFα, by Cycloprodigiosin Hydrochloride

cPrG-HCl is a physiologically active substance which shows a marked anticancer effect and immunosuppressive effect by inducing apoptosis in various tumor cells and activated lymphocytes. In order to reveal the mechanism of the anticancer effect and immunosuppressive effect of the cPrG-HCl, analysis was made on the effect of cPrG-HCl on the signal transduction system of tumor necrosis factor TNFα, for which a detailed mechanism of apoptosis induction has been revealed.

TNFα induces apoptosis by binding to the TNF receptor (TNFR) on the cell membrane to activate caspase through TRDD or FADD. On the other hand, the TNFR induces phosphorylation and degradation of the NF-κB inhibitory protein, IκB, by activating IKK (IκB kinase). Due to the degradation of the IκB, NF-κB is translocated to the nucleus to induce expression of genes such as the inhibitor of apoptosis protein (IAP) gene. Namely, signals for both “life” and “death” in the cells are simultaneously activated by TNFα and thus the balance of these signals determines the fate of the cells. The method and the result of the experiment are described as follows (Kamata, K., Okamoto, S., Oka, S., Kamata, H., Yagisawa, H., Hirata, H. (2001) FEBS Lett. 507:74-80).

The plasmids, pRKF-IKKα and pRKHA-IKKβ have been described previously (Oka, S., Kamata, H., Kamata, K., Yagisawa, H. and Hirata, H. (2000) FEBS Lett., 472, 196-202). pRKF-IKKαEE and pRK-HA-IKKβEE encode constitutively active mutants of IKKα, in which 176 and 180 serine residues in an activation loop are replaced by glutamic acid residues, and IKKβ, in, which 177 and 181 serine residues are replaced by glutamic acid residues, respectively. A reporter plasmid pIG3Luc for NF-κB encodes triple NF-κB binding site sequences (AGCTTCAGAGGGGACTRANSCRIPTIONCCGAGAGG) (SEQ ID NO: 2) followed by the luciferase gene. A control plasmid, pBKRLuc, was obtained from Toyobo (Toyo B-Net Co., Japan). pcDNAF-MEKK1, pRKF-p65, pEFBOSHA-Rac1-DA, and pEFBOSHA-Cdc42-DA, are the expression plasmids for MEKK1, the NF-κB subunit p65, constitutively active Rac1 and Cdc42, respectively.

Cell culture: HeLa cells were cultured in Dulbecco's modified Eagle's medium supplemented with 10% fetal bovine serum and 50 μg/ml kanamycin at 37° C. in 5% CO2 in air.

Assay for apoptotic processes: Cell viability was determined by dye exclusion assay using 0.1% trypan blue. DNA fragmentation was analyzed as described previously (Kawauchi, K. et al. (1997) Biochem. Biophys. Res. Commun. 237, 543-547). In the caspase-3 assay, cells were disrupted by freeze-thaw in an extraction buffer (50 mM Tris-HCl, pH7.4, 1 mM EGTA, 5 mM EDTA, 5 mM MgCl₂, 1 mM APMSF, 10 μg/ml pepstatin, 10 μg/ml leupeptin, and 1 mM DTT). Then the suspension was centrifuged at 15,000 rpm for 20 min, and the supernatants obtained were incubated at 37° C. for 60 min in a reaction buffer (50 mM Tris-HCl, pH7.4, 1 mM EDTA, 10 mM EGTA) with 20 μM of the fluorogenic substrate DEVD-AFC (Peptide Institute, Inc., Japan). Cleavage of the substrates was quantified by measuring the fluorescence of 7-amino-4-trifluomethyl coumarin released using a spectrofluorometer (Shimadzu RF5000) with excitation at 380 mn and emission at 460 nm.

Luciferase gene reporter assay and Kinase assays of IKK: HeLa cells were transfected with the reporter plasmids described above using Lipofectin (GIBCO BRL). After 24-48 hr, the luciferase activity was determined using the Luciferase Assay System (Promega). The kinase activity of IKKα and IKKβ were analyzed by immune complex kinase assay as described previously (Oka, S., Kamata, H., Kamata, K., Yagisawa, H. and Hirata, H. (2000) FEBS Lett. 472, 196-202). The substrates was using GST-IκBα(1-55) or GST-p65(399-551).

Electrophoretic mobility shift assay (EMSA): Nuclear extracts were prepared by the method of Schreiber, et al (Schreiber, E., Matthias, P., Muller, M. M. and Schaffner, W. (1989) Nucleic Acids Res. 17, 6419). Briefly, cells were solubilized with a buffer (10 mM HEPES-NaOH, pH 7.9, 10 mM KCl, 0.1 mM EDTA, 0.1 mM EGTA, 0.4% NP-40, 0.5 mM PMSF, 1 μg/ml aprotinin, 1 μg/ml leupeptin, 1 μg/ml pepstatin, 1 mM DTT), and then centrifuged at 10,000 rpm for 15 min. The pellets were resuspended in a buffer (20 mM HEPES-NaOH, pH 7.9, 400 mM NaCl, 1 mM EDTA, 1 mM EGTA, 1 mM PMSF, 1 μg/ml aprotinin, 1 μg/ml leupeptin, 1 μg/ml pepstatin, 1 μg/ml chymostatin, 1 μg/ml antipain, 1 mM DTT), and then centrifuged at 15,000 rpm for 15 min. The supernatant was used as the nuclear extract. ³²P-labeled DNA probes of the NF-κB binding site (AGCTTCAGAGGGGACTRANSCRIPTIONCCGAGAGGTCGA) (SEQ ID NO: 3) were prepared as described previously (Tanaka, C., Kamata, H., Takeshita, H., Yagisawa, H. and Hirata, H. (1997) Biochem. Biophys. Res. Commun. 232, 568-573). The nuclear extract (10 μg of protein) was incubated with the ³²P-labeled probes (100,000 cpm) in 20 μl of buffer (20 mM HEPES-NaOH, pH 7.9, 5% glycerol, 1 mM EDT, 100 μg/ml poly (dI-dC)) for 20 min at room temperature. The samples were electrophoresed on an 8% polyacrylamide gel and analyzed by an Imaging plate (Fuji Film).

Western blot analysis: Cells were solubilized with a buffer (20 mM Tris-HCl, pH7.4, 10 mM EGTA, 10 mM MgCl₂, 1 mM benzamidine, 60 mM β-glycerophosphate, 1 mM Na₃VO₄, 20 mM NaF, 1 mM APMSF, 50 u/ml aprotinin, 20 μg/ml pepstatin, 20 μg/ml leupeptin, 1 mM DTT, and 1% Triton X-100), and then centrifuged at 15,000 rpm for 20 min. The supernatants were used as the cell extracts. The cell extracts and the nuclear extracts were subjected to SDS-polyacrylamide gel electrophoresis, and transferred to an ECL membrane (Amersham Pharmacia). Western blot analysis was performed using a Western Blotting Detection System (Amersham-Pharmacia) according to the manufacturer's instructions.

cPrG-HCl Induces Apoptosis in TNFα-Induced HeLa Cells

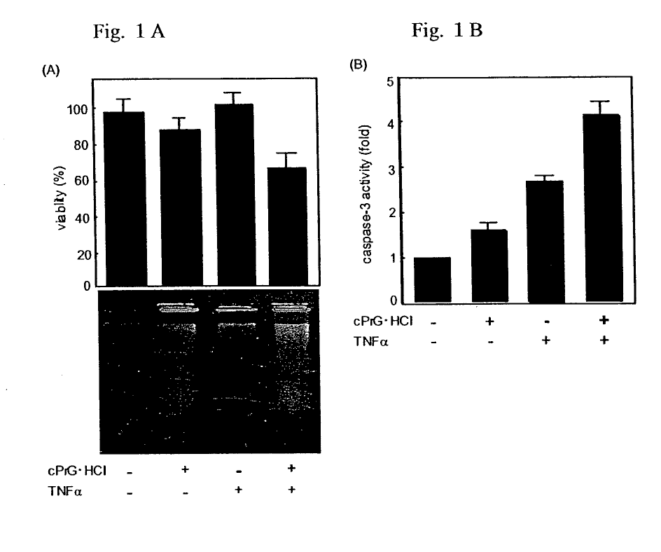

HeLa cells were treated with TNFα and cPrG.HCl and the effect of cPrG.HCl on apoptosis was examined by measuring DNA fragmentation and survivability. Although apoptosis was hardly induced by treating the cells with 1 μM cPrG-HCl, marked DNA fragmentation was induced when cells were treated with cPrG-HCl together with TNFα (FIG. 1A). Further, when activity of caspase-3, which plays a major role in apoptosis induction, was measured, the caspase-3 activation was markedly induced by treating with cPrG-HCl and TNFα (FIG. 1B). These results revealed that cPrG-HCl enhanced TNFα induced apoptosis. Further, cPrG-HCl presumably enhanced the effect of the “death” signal by suppressing the “life” signal by TNFα.

cPrG-HCl Suppresses TNFα- and PMA-Induced Activation of NF-κB without Attenuating Upstream Cellular Signaling Pathways

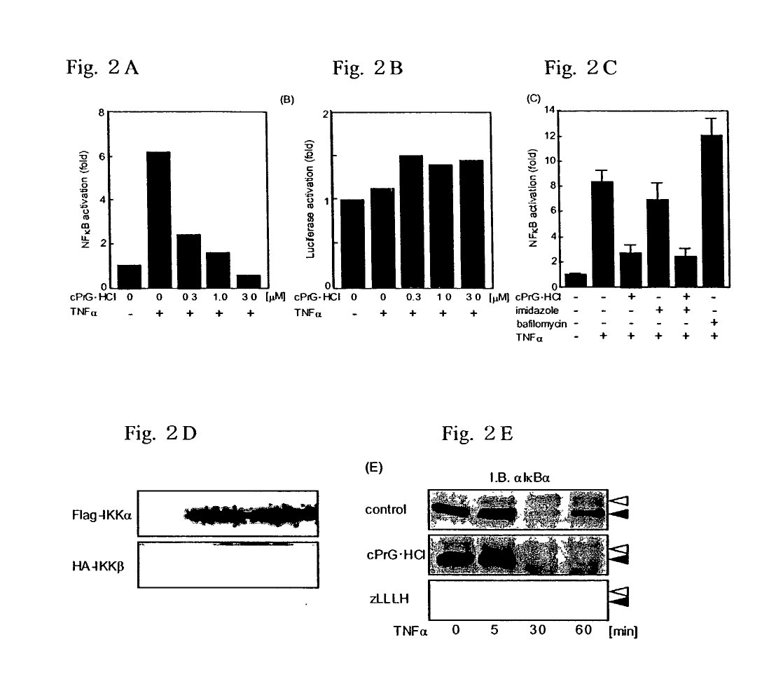

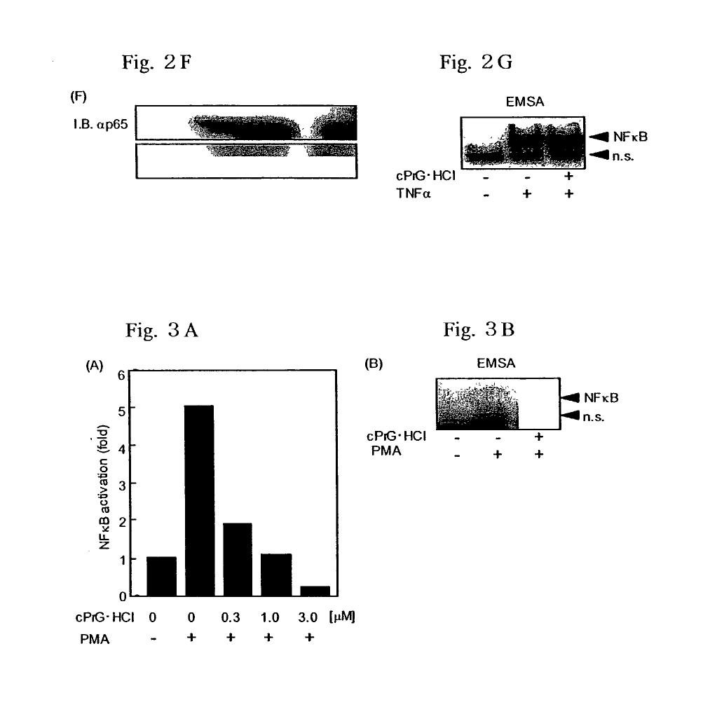

The gene expression induction by NF-κB activation plays the most important role among “life” signals by TNFα. Therefore, the NF-κB reporter gene was introduced into the cells to analyze the effect of cPrG-HCl by luciferase assay. The result revealed that cPrG-HCl inhibited the gene expression induction by NF-κB (FIG. 2A).

cPrG-HCl is an agent to induce acidification of intracellular pH by cotransportation of H⁺ and Cl⁻ via a biological membrane. So far the apoptosis induction by cPrG-HCl was considered to be associated with the acidification of intracellular pH. However, when the effect of cPrG-HCl on NF-κB was measured by adding a cell membrane permeable weak base, imidazole, to a medium so that no acidification of intracellular pH be induced, it was revealed that cPrG-HCl suppressed the TNFα-induced NF-κB activation even in the presence of imidazole (FIG. 2C). Further, TNFα-induced NF-κB activation was not suppressed when acidification of intracellular pH was induced by a V-ATPase inhibitor, bafilomycin (FIG. 2C). These results showed that acidification of intracellular pH was not associated with the suppression of NF-κB activity by cPrG-HCl.

In order to find out the mechanism of NF-κB suppression by cPrG-HCl, the effect on the signal transduction system, which functions upstream of NF-κB, was analyzed. First, the effect of cPrG-HCl on IKK activity was examined. The IKK activation was induced by TNFα stimulation but cPrG-HCl did not suppressed this activation (FIG. 2D). Next, the effect of cPrG-HCl on phosphorylation and degradation of IκB was analyzed by Western blotting using anti-IκB antibody (FIG. 2E). In FIG. 2E, black arrows indicate IκBα and white arrows indicate phosphorylated IκBα. At 1 hour after TNFα stimulation, the phosphorylation of IκBα was induced but cPrG-HCl did not suppress this phosphorylation. Further, at 30 minutes after TNFα stimulation, IκBα was decomposed by proteasome but cPrG-HCl also did not suppress this degradation (FIG. 2F). Namely, cPrG-HCl suppressed NF-κB activation without suppressing the steps up to IκB degradation. Accordingly, nuclear translocation of NF-κB was analyzed by Western blotting using p65 and p50 which are components of NF-κB (FIG. 2F). It was revealed that p65 and p50 in the nucleus were confirmed to be increased in response to the TNFα stimulation, but cPrG-HCl did not inhibit this increase and accordingly the nuclear translocation of NF-κB was not inhibited. Further, the state of NF-κB activation in the nucleus was analyzed by EMSA (electrophoretic mobility shift assay) (FIG. 2G). In FIG. 2G, “n. s.” means nonspecific band. Although DNA binding activity of NF-κB was increased by TNFα stimulation, cPrG-HCl did not suppress this activation. PMA stimulation also induced transcriptional activation of NF-κB, but cPrG-HCl inhibited neither the nuclear translocation of NF-κB nor DNA binding activation by PMA (FIG. 3A and FIG. 3B).

cPrG-HCl Suppresses the NF-κB-Dependent Gene Expression Induced by Rac1, Cdc42, MEKKI, IKKα, IKKβ and p65

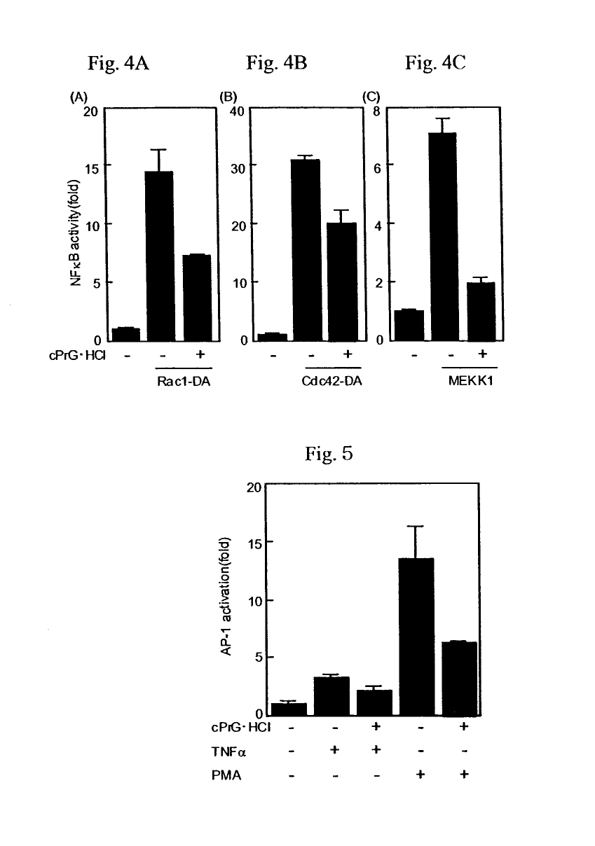

NF-κB is translocated into the nucleus also by activation with low-molecular-weight G proteins Rac1 and Cdc42 or MEKK1 and induces gene expression. An activated Rac1, Cdc42 or MEKK1 expression plasmid was introduced together with the NF-κB reporter gene into cells and the effect of cPrG-HCl on NF-κB activation was measured by luciferase assay (FIG. 4A and FIG. 4B). The NF-κB activation was markedly induced by Rac1, Cdc42 and MEKK1, but cPrG-HCl suppressed this activation. From these results, cPrG-HCl presumably inhibited the gene expression at the level of transcriptional activation after NF-κB is translocated into the nuclear and bound to the DNA, without inhibiting the signal transduction system functioning upstream of the NF-κB.

Thus, the NF-κB reporter gene was introduced into HeLa cells and the effect of cPrG-HCl on NF-κB transcriptional activation was analyzed by luciferase assay, which revealed that cPrG-HCl concentration-dependently suppressed the TNFα-induced NF-κB activation (FIG. 2A). On the other hand, since cPrG-HCl did not inhibit expression of the CMV promoter-dependent luciferase gene used as a control, presumably cPrG-HCl specifically suppressed the NF-κB-dependent transcriptional activation (FIG. 2B). Cell viability was slightly decreased and DNA fragmentation was also observed by cPrG-HCl treatment (FIG. 1A). On the other hand, the decrease in cell viability and the DNA fragmentation were hardly observed when stimulation was by TNFα alone (FIG. 1A). In contrast, when cPrG-HCl was present upon the TNFα stimulation, the cell viability decreased down to about 65% and significant DNA fragmentation was confirmed (FIG. 1A). Thus, it was revealed that cPrG-HCl enhanced TNFα-induced apoptosis. Further, marked synergistic caspase-3 activation was observed with cPrG-HCl and TNFα (FIG. 1B).

Although various drugs have been so far reported to suppress NF-κB, most of them act on upstream of the signal system of NF-κB, such as IKK. In contrast, cPrG-HCl had a unique characteristic to act at the level of transcription. Further, cPrG-HCl can effectively induce apoptosis in cancerous cells by the synergistic effect of two different actions, i.e., the acidification of intracellular pH and the suppression of NF-κB.

### EXPERIMENTAL EXAMPLE 2

Suppression of Rb Phosphorylation

A431 cells were treated with cPrG-HCl (1 μM) for 24 hours and the extract prepared from the cells was analyzed by Western blotting using anti-Rb antibody.

As a result, a band of phosphorylated Rb (112 KDa) was detected without stimulation, whereas a band of 110 KDa was detected when treated with cPrG-HCl showing that the phosphorylation of Rb was suppressed (FIG. 10).

### EXPERIMENTAL EXAMPLE 3

Suppression of NF-κB Phosphorylation by Cycloprodigiosin Hydrochloride

cPrG-HCl is a H⁺/Cl⁻ symporter or a physiologically active substance which induces acidification of intracellular pH. Independently of the acidification of intracellular pH, cPrG-HCl suppresses the activation of transcription factor NF-κB which plays an important role in cell growth and immune response reactions. Its point of action is the transcriptional activation step after DNA binding. Analysis of the effect of cPrG-HCl on phosphorylation of the NF-κB component p65, which is important in the transcriptional activation step, was carried out as follows.

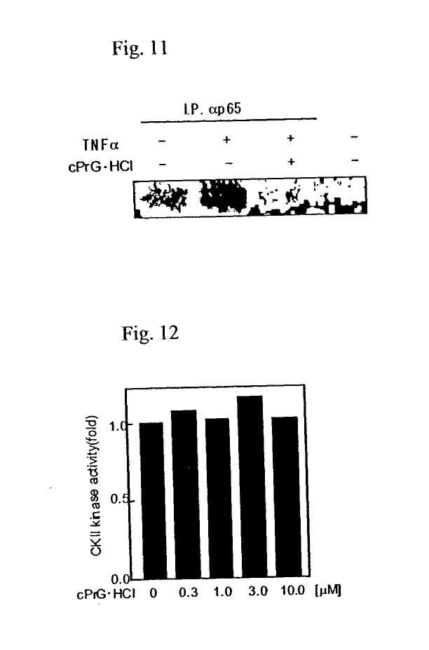

The effect of cPrG-HCl on p65 phosphorylation was examined in a cell labeling experiment using [³²P] orthophosphoric acid. The result revealed that cPrG-HCl suppressed TNFα-induced phosphorylation (FIG. 11). In FIG. 11, cells labeled with the radioisotope were analyzed by an Image Analyzer (as described above for kinase assay); from left to right, the first lane shows no stimulation, the second lane shows TNFα, the third lane shows TNFα+cPrG, and the fourth lane shows no stimulation. In the first, second and third lanes, immune precipitation was carried out with p65 antibody and protein A-sepharose. The fourth lane was a negative control with protein A-sepharose alone. A band was found only in the second, which showed that cPrG-HCl suppressed the p65 phosphorylation.

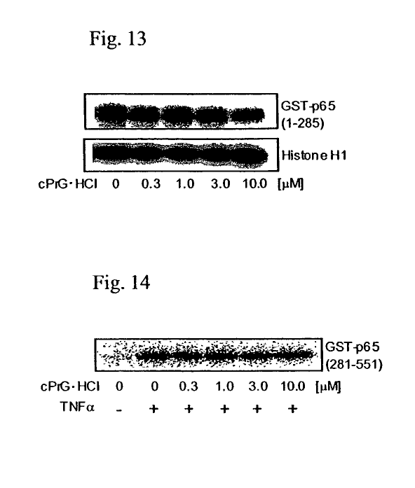

Accordingly, in order to identify the p65 kinase which cPrG-HCl suppressed, the effect of cPrG-HCl on PKA (FIG. 13), casein kinase II (CKII) (FIG. 12), and IκB kinase (IKK) (FIG. 14) was studied in vitro. The result showed no suppressive effect. It was thus suggested that cPrG-HCl suppressed through a certain signal or suppressed an unknown kinase other than those mentioned above.

### EXPERIMENTAL EXAMPLE 4

Suppressive Effect on AP-1 Transcriptional Activity

In the same manner as described in Experimental example 1, HeLa cells were transfected with the AP-1 reporter gene plasmid pAPLuc. After 20 hours, the cells were pretreated with cPrG-HCl (1 μM) for 1 hour and then stimulated with TNFα (20 ng/ml) or PMA (1 μg/ml) for 4 hours, after which, luciferase activity was measured. Each activity was corrected for the amount of protein. The AP-1 transcriptional activity was also significantly suppressed by cPrG-HCl (FIG. 5).

In addition, in order to reveal the mechanism of the suppression of AP-1 transcriptional activation, the effect of cPrG-HCl on upstream kinases, i.e., JNK and p38, was analyzed. Further, relationship between the suppression of AP-1 transcriptional activation by cPrG-HCl and the acidification of intracellular pH was studied. The result suggested that the AP-1 suppression by cPrG-HCl was not caused due to the intracellular acidification since cPrG-HCl suppressed PMA-induced AP-1 transcriptional activation even in the presence of imidazole, whereas the intracellular acidification by cPrG-HCl be inhibited by adding the cell membrane permeable weak base to a medium. Further, analysis by the EMSA method showed that cPrG-HCl exhibited no inhibitory effect on DNA binding activity of AP-1. Further, cPrG-HCl exhibited inhibitory effect on neither JNK nor p38 kinase. These results suggested that cPrG-HCl did not inhibit the kinases which function upstream of AP-1, but suppressed the step of transcriptional activation by AP-1. Since cPrG-HCl suppressed the step of transcriptional activation similarly with NF-κB, cPrG-HCl presumably inhibited a transcription mechanism common to these factors.

### EXPERIMENTAL EXAMPLE 5

Cell-Type Specificity of Suppression of NF-κB and AP-1 Activation

In order to find out whether the suppression of NF-κB and AP-1 activation was specific to HeLa cells used above, analysis was made with various kind of cells. The cells used were U373 cells (human brain tumor cells), 293 cells (human embryo-derived kidney cells), COS7 cells (African green monkey kidney-derived cells), and MKN45 cells (human gastric tumor cells).

The NF-κB reporter plasmid pIG3Luc was incorporated into cells by transfection. After 24 hours, the cells were pretreated with cPrG-HCl at a specified concentration for 1 hour and then stimulated with TNFα (20 ng/ml) or PMA (1 μg/ml) for 4 hours, after which luciferase activity was measured. Each activity was corrected for the amount of protein.

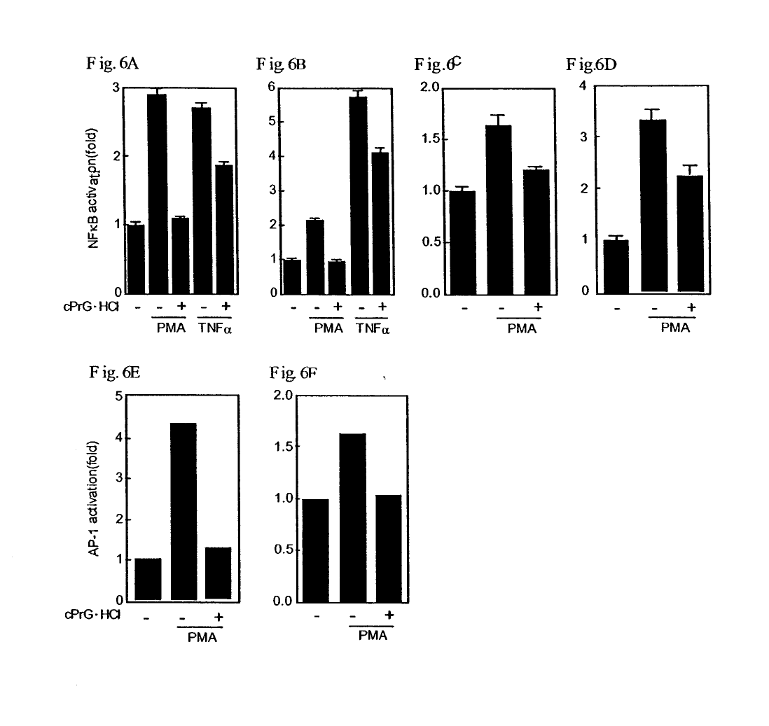

The result showed that cPrG-HCl suppressed the NF-κB and AP-1 transcriptional activation in all the cells tested (FIGS. 6A to 6F).

### EXPERIMENTAL EXAMPLE 6

Effect of cPrG-HCl on IL-8 Gene Expression

cPrG-HCl was shown to suppress NF-κB and AP-1 in the examples above. Now in order to analyze the effect of cPrG-HCl on IL-8 gene expression which is controlled by transcription factors such as NF-κB and AP-1, luciferase assay was carried out using a reporter plasmid (pIL-8Luc) in which the luciferase gene is bound downstream of the IL-8 promoter region. In the IL-8 promoter, NF-κB, AP-1, and NF-IL6 binding sequences are present.

The pIL-8Luc was incorporated into HeLa cells by transfection. After 24 hours, the cells were pretreated with cPrG-HCl at a specified concentration for 1 hour and then stimulated with TNFα (20 ng/ml) for 4 hours, after which luciferase activity was measured.

The result showed that cPrG-HCl markedly suppressed IL-8 promoter-dependent transcriptional activity induced by TNFα (FIG. 7).

It was shown that therapeutic effect can be attained by using cPrG-HCl for treating cancerous cells in which NF-κB or AP-1 is being activated, or by activating cancerous cells with NF-κB or AP-1 and then administering cPrG-HCl.

Further, the result showed that cPrG-HCl suppressed not only CRE-dependent transcriptional activation but also NF-κB and AP-1 transcriptional activation and thus could be a strong inhibitor of the production of inflammatory cytokines including IL-8.

### EXPERIMENTAL EXAMPLE 7

Effect of cPrG-HCl on CRE Binding Factor

cPrG-HCl inhibits gene expression which depends on transcription factor and binding to the CRE sequence and cytokine or chemoatractant gene expression which depends on NF-κB or AP-1, and thus makes it possible to treat autoimmune diseases including rheumatism.

Here in order to analyze the effect of cPrG-HCl on the CRE binding factor, luciferase assay was carried out using a reporter plasmid (pCRELuc) in which the luciferase gene is linked downstream of the CRE sequence.

The pCRELuc was transfected in COS-7 cells. After 24 hours, the cells were pretreated with cPrG-HCl at a specified concentration for 1 hour and further stimulated with phorbol ester myristate acetate PMA (1 μg/ml) for 4 hours, after which luciferase activity was measured.

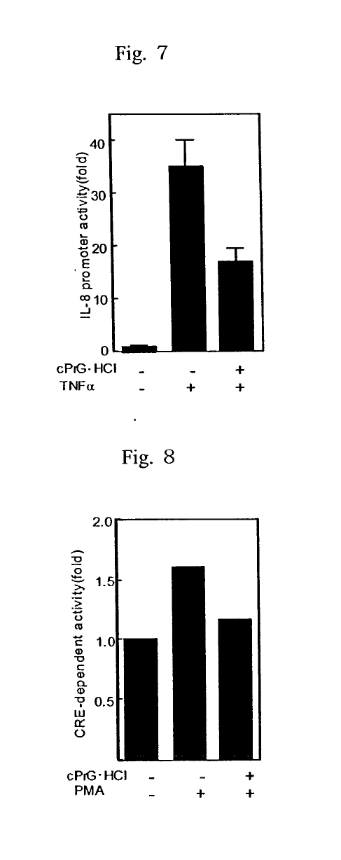

The result showed that cPrG-HCl markedly suppressed PMA-induced CRE-dependent transcriptional activity (FIG. 8).

### EXPERIMENTAL EXAMPLE 8

Effect of cPrG-HCl on Upstream Kinase

Next, in order to analyze the effect of cPrG-HCl on upstream kinase, the effect on JNK and p38 kinase activity and the effect on p38 phosphorylation were analyzed.

HeLa cells were pretreated with cPrG-HCl (1 μM) for 1 hour and then stimulated with TNFα (20 ng/ml) or PMA (1 μg/ml) for 15 minutes, and JNK recovered by an immune precipitation method was then reacted with [γ-³²P]-ATP and a substrate GST-c-Jun, after which SDS-PAGE was carried out and the resulting gel was analyzed by an imaging analyzer. Further, HeLa cells were pretreated with cPrG-HCl (1 μM) for 1 hour and then stimulated with TNFα (20 ng/ml) for 15 minutes, a cell extract was recovered and subjected to SDS-PAGE and Western blotting was carried out using p38 phosphorylation antibody.

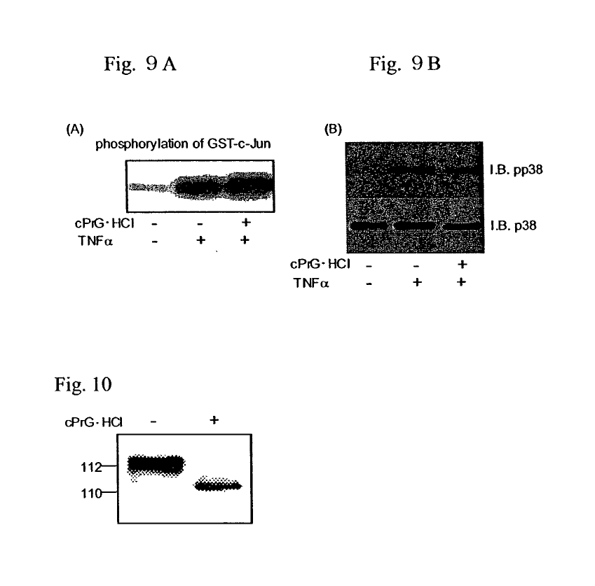

The result showed that cPrG-HCl suppressed neither JNK nor p38 kinase (FIG. 9A and FIG. 9B).

What is claimed is:

1. A method of treating neoplastic cells, comprising the steps of;
examining whether a potential subject has neoplastic cells in which Rb is present and NF-κB and/or AP-1 are constitutively being activated; and
administering cycloprodigiosin (cPrG) or a pharmaceutically acceptable salt thereof in an amount effective to suppress Rb phosphorylation and to suppress transcriptional activity of NF-κB and/or AP-1 which are constitutively being activated, to the potential subject to treat the neoplastic cells.

2. The method according to claim 1, wherein said pharmaceutically acceptable salt is hydrochloride.

3. The method according to claim 1, wherein said neoplastic cells are tumor cells selected from the group consisting of leukemic cells, testic cancer cells, melanoma, breast cancer, and hepatoma.

4. The method according to claim 1, wherein said neoplastic cells are treated in the presence of tumor necrosis factor.

5. The method according to claim 4, wherein the tumor necrosis factor is TNFα.

6. The method according to claim 1, wherein the tumor necrosis factor is administered together with cycloprodigiosin.

7. A method of treating neoplastic cells, comprising the steps of:
examining whether a potential subject has neoplastic cells in which Rb is present and NF-κB and/or AP-1 are not constitutively being activated; and
administering cycloprodigiosin or a pharmaceutically acceptable salt thereof in an amount effective to suppress Rb phosphorylation, to the potential subject to treat the neoplastic cells.

8. A method of treating an autoimmune disease, comprising the steps of:
examining whether a potential subject has an autoimmune disease caused by gene expression depending on binding to a CRE sequence; and
administering cycloprodigiosin or a pharmaceutically acceptable salt thereof in an effective amount to the potential subject to treat said autoimmune disease.

9. The method according to claim 8, wherein said pharmaceutically acceptable salt is hydrochloride.

10. The method according to claim 8, wherein said gene binding to the CRE sequence is a gene of an active factor selected from the group consisting of cytokines, chemoatractants and cell adhesive factors.

11. The method according to claim 8, wherein said autoimmune disease is rheumatism.
